(19) United States
(12) Patent Application Publication
(10) Pub. No.: US 2004/0258748 A1
(43) Pub. Date: Dec. 23, 2004

(54) Process for the preparation of fast dissolving dosage form

(76) Inventors: Ashish Madan, New Delhi (IN); Anupam Trehan, New Delhi (IN); Vinod Kumar Arora, New Delhi (IN)

Correspondence Address: Jayadeep R Deshmukh, Ranbaxy Inc, 600 College Road East, Suite 2100, Princeton, NJ, 08540, US

(21) Appl. No.: 10/490,398

(22) PCT Filed: Sep. 25, 2002
(86) PCT No.: PCT/IB02/03969

(30) Foreign Application Priority Data
Sep. 25, 2001 (IN) 981/DEL/2001

(51) Int. Cl. A61K 9/20; B27N 3/00

(52) U.S. Cl. 424/464; 264/109

(57) ABSTRACT

The present invention relates to a process for the preparation of fast dissolving dosage form, such as tablet, which disintegrates quickly in the mouth.

### FIELD OF THE INVENTION

The present invention relates to a process for the preparation of fast dissolving dosage form, such as tablet, which disintegrates quickly in the mouth.

### BACKGROUND OF THE INVENTION

With the increase in average human life span, drug administration for elderly patients has become more important. Old age is normally accompanied by the onset of degenerative pathologies involving difficulties in coordination and in swallowing the conventional dosage forms such as tablets or capsules. Swallowing problems are also present, in other population groups, such as children. Need for dosage forms having quick onset of action is particularly felt even for those patients who do not have swallowing problems. Similarly, in cases of motion sickness, sudden episodes of allergic attacks, or coughing, the swallowing becomes difficult. Fast dissolving or disintegrating tablets provides the solution to such problems. These tablets disintegrate quickly in saliva or water.

Different techniques are used to prepare fast dissolving tablets. Most of these techniques aim at making porous particles/granules or tablets, so that mouth dissolving time can be reduced. Freeze drying, spray drying, sublimation, disintegrant addition, shearform technology and tablet molding are examples of such techniques.

U.S. Pat. Nos. 4,305,502; 4,371,516 and 5,738,875 describe the use of freeze-drying process to prepare an amorphous, porous structure, which dissolves rapidly. However, such formulations are very expensive and require sophisticated technologies and methods from the production point of view. The tablets prepared by this method are difficult to handle and require special packaging.

U.S. Pat. Nos. 5,587,180; 5,635,210; 5,595,761 and 5,807,576 describe the spray drying technique to prepare highly porous particulate support matrix, which is then mixed with an active agent and compressed to form a tablet. This technique is quite expensive and cannot be used for drugs which become unstable on losing their crystalline structure.

The sublimation technique described in U.S. Pat. Nos. 3,885,206; 4,134,943 and 5,762,961 use mannitol and camphor as pore forming agents. The tablets prepared by this method disintegrate within 10 to 20 seconds.

U.S. Pat. Nos. 4,134,943 and 5,720,974 describe the use of water as a pore forming agent. A mixture containing an active ingredient and a carbohydrate is moistened with water and compressed into tablets. The removal of water yields highly porous tablets. However, this process is not practically feasible. The high water content in the granules makes the compression difficult.

U.S. Pat. No. 6,149,938 describes that mouth-soluble, rapidly disintegrating tablets can be prepared by fluidized bed granulating an aqueous solution of a water-soluble or water-dispersible polymer in a polyalcohol, optionally in mixture with other solid components.

Disintegrant addition is another method of making fast dissolving tablets. Use of effervescent mixture, which generally consists of an acid and a gas-generating base as a disintegrant for the preparation of porous granulates, or particles is also known.

Different processes have been used to prepare porous granulates of effervescent mixture suitable to the preparation of fast dissolving tablets.

U.S. Pat. No. 3,207,824 describes a process for preparing effervescent granules which involves mixing the dry powders together to form a dry mix, adding a small amount of water which starts the effervescence reaction so that a workable mass is obtained; quickly drying the mass in ovens or heated dishes to stop the reaction; and grinding the mass under the dry conditions to form powder or granules.

U.S. Pat. No. 3,401,216 describes a technique consisting of suspending a dry mixture of the acid and the base in powder form in the stream of gas, thereby forming a constantly agitated “fluidized bed” and introducing into this bed just so much of a fluid which causes said chemical ingredient to react to only a limited extent.

French Patent Nos. 7112175 and 7135069 describe a technique which involves the careful humidification of sodium bicarbonate by a very small quantity of demineralized water, then addition of citric acid and optionally a binding agent, in a mixture, which starts off the reaction of the bicarbonate on the citric acid. This mixture is pre-dried in a fluid bed dryer by blowing hot air, which interrupts the reaction. The final drying is again done in fluid bed dryer by blowing hot air.

This technique has a drawback of necessitating the transfer of the filler, from the mixer to the drier. Consequently, the effervescent reaction triggered off in the mixer cannot be mastered with total precision as its interruption, in the drier, depends on the time for emptying and transferring the filler towards the drier.

U.S. Pat. No. 5,437,873 describes a process for the preparation of superior tasting pharmaceutical composition having porous particles. Stiochiometeric amounts of an appropriate base and an appropriate acid are mixed and compressed in a press to form a compact. The compact is then milled to form an evenly distributed stiochiometeric mixture of the base and the acid. A pharmacologically active is then added to the mixture and wet granulated. The wet granulated material is then dried whereby the applied heat and the water cause the acid and the base to react releasing gas from the wet granulation to form porous particles. The porous particles are then milled to form powder, which is then compressed to form a tablet.

EP 494972 patent describes effervescent tablets suitable to the direct oral administration, i.e. without a previous development of the effervescence in water, consisting of microcapsules containing the active ingredients and an amount of effervescent agents sufficient to promote the release of the microgranules when ingested and to give a “fizzing” sensation when in contact with the buccal mucosa to the patient. Such a preparation technique yields tablets having friability values higher than those involving the humid granulation of the mixture to be pressed. Tablets prepared by this technique have higher dissolution time.

All the above mentioned prior art processes, except the freeze drying and sublimation techniques describe the preparation of porous particles or granules, which are then compressed to form the fast dissolving tablets. However, due to compression pressure these porous particles/granules undergo rearrangement to form a less porous structure. This decrease in porosity results in increased dissolution/disintegration time. So the whole purpose of making fast dissolving tablets by using porous particles/granules gets defeated once compression pressure is applied to them.

### SUMMARY OF THE INVENTION

The present invention addresses the drawbacks and problems associated with currently available technologies. It avoids the use of expensive and non-conventional equipment like freeze dryer or spray dryers.

The present invention relates to a process for the preparation of fast dissolving/disintegrating tablets wherein the porosity is produced by in-situ gas generation through moisture activation of the tablets comprising effervescent mixture.

The present invention provides tablets with short dissolution/disintegration time as porosity is achieved in the tablet rather than by making porous particles or granules. When such tablets are placed in the oral cavity, saliva quickly penetrates into the pores to cause rapid disintegration/dissolution. The tablets prepared by the process of present invention dissolve in saliva in preferably less than 20 seconds. The present invention has a further advantage as markedly lower amounts of effervescent mixture than those usually employed in conventional effervescent tablets can be used. The use of lower effervescent mixture concentration gives the advantage of better taste and pleasant mouth feel against the abrasiveness and burning sensation experienced with higher concentrations.

Furthermore, the process of the present invention is simple and cost effective. It can easily be carried out in a traditional effervescent tablet plant. The tablets prepared by the process of the present invention maintain their structural integrity and can be handled and packed as conventional effervescent tablets.

### DETAILED DESCRIPTION OF THE INVENTION

The present invention provides a process of preparing fast dissolving dosage form for oral administration, comprising the steps of

a) compressing a blend comprising a pharmaceutical active ingredient and effervescent mixture comprising an acid source and a base to produce a tablet, and

b) subjecting said tablet to moisture activation.

The term “moisture activation” means activating an acid base reaction by providing moisture. The moisture causes the acid and the base present in the tablet to effervesce, the gas produced tries to escape forming pores in the tablets. The moisture activation can be done by subjecting the tablets comprising the effervescent mixture to either controlled humidity or controlled heating.

The moisture activation by controlled humidity can be achieved by subjecting the tablets containing the effervescent mixture to careful humidification, which starts off the reaction of the base and acid. This can easily be done by keeping the tablets in relative humidity chamber at a percentage relative humidity of 20 to 100% depending on the temperature.

An alternative process for moisture activation is by controlled heating. In this method, tablets containing the effervescent mixture are heated to liberate water of crystallization. The water thus liberated initiates the acid and base reaction, releasing carbon dioxide which generates pores. For this method, the presence of at least one ingredient having water of crystallization is required. Heating can be done as such or under vacuum. The heating temperature would vary according to the ingredient from which the water of crystallization is to be liberated.

The tablets comprising an effervescent mixture can be prepared by any method known in the art. The effervescent mixture consists of an acid source and a base.

The acid source can be an acid, anhydride or an acid salt. The acid is selected from the group consisting of citric, tartaric, malic, fumaric, adipic, succinic, and alginic acids. The acid salts include dihydrogen phosphate, disodium dihydrogen phosphate, and citric acid salts.

The bases can be solid carbonates of salts such as sodium carbonate, sodium bicarbonate, potassium bicarbonate, potassium carbonate, magnesium carbonate, sodium glycine carbonate, L-lysine carbonate, arginine carbonate and amorphous calcium.

The amount of effervescent mixture is from 1% to 35% by weight of the total composition, preferably 15-20%.

Since the tablets of the present invention consist of an intimate mixture of components which are highly reactive in the presence of moisture, it is apparent that the control of humidity is an extremely important factor in the production of commercially acceptable and stable tablets. Uncontrolled humidity or prolonged exposure to moisture, or even excessive moisture content, will cause the base and the acid to react. Since this reaction not only forms salt and carbon dioxide but water as well, the decomposition reaction is progressive. Therefore, preferably the acid base reaction is interrupted by applying vacuum. The vacuum is applied until the entire moisture is removed.

The active ingredient may be selected from the pharmaceuticals but may also include vitamins, minerals or dietary supplements. Pharmaceuticals may include antacids such as omeprazole, non-steroidal anti-inflammatory drugs such as rofecoxib and nimesulide, steroidal anti-inflammatory drugs such as betamethasone, anti-psychotic drugs such as olanzapine, hypnotic drugs such as alprazolam, antiepileptic drugs such as sodium valproate, antiparkinsonism drugs such as levodopa, hormone drugs such as progestin, analgesic drugs such as aspirin, serotonin 5HT receptor antagonists such as ondansetron, diuretic drugs such as sulphamethoxazole, H2 receptor antagonists such as ranitidine hydrochloride, antiarrhythmic drugs such as pindolol, cardiotonic drugs such as digitoxin, coronary vasdilators such as nitroglycerin, calcium antagonists such as diltiazem hydrochloride, antihistaminic drugs such as fexofenadine hydrochloride, antibiotics such as doxycycline, antitumor drugs such as actinomycin, antidiabetic drugs such as metformin, gout treating drugs such as allopurinol, antiallergic drugs such as loratadine, antihypertensive drugs such as quinapril, central nervous system acting drugs such as indeloxazine hydrochloride, antispasmodic drugs such as butylscopolamine, antihyperlipidemic drugs such as simvastatin, bronchodilators such as salbutamol, α-adrenergic receptor blockers such as tamsulosin hydrochloride, osteoporosis treating drugs such as sodium alderonate, antifungal drugs such as fluconazole, antiviral drugs such as lamivudine, drugs for erectile dysfunction such as sildenafil and antidepressant such as sertraline.

The invention is further illustrated by the following examples but they should not be construed as limiting the scope of this invention in any way.

### EXAMPLE 1

Rofecoxib Mouth Soluble Tablets (50 mg Strength)

| Ingredients | Mg/Unit |
|---|---|
| Rofecoxib | 50 |
| Polyvinylpyrrolidone | 0.375 |
| Water | qs |
| mannitol | 172.226 |
| Microcrystalline cellulose | 50 |
| L-hydroxypropyl cellulose | 20 |
| Sodium bicarbonate | 48 |
| Citric acid (anhydrous) | 36 |
| Aspartame | 11.6 |
| Colloidal Silicon dioxide | 2.0 |
| Mango Flavour | 4.166 |
| Banana Flavour | 0.833 |
| Magnesium stearate | 4.8 |
| Total | 400.00 |

Method

1. Rofecoxib (granulated), mannitol, sodium bicarbonate (preheated at 80° C. for 1 hour), L-hydroxypropyl cellulose, microcrystalline cellulose, Aspartame, colloidal silicon dioxide, Mango flavour, Banana flavour are sifted through 44 BSS sieve.

2. The blend is mixed for 10 minutes in a double cone blender.

3. Citric acid (preheated at 80° C. for 1 hour) is sifted through 100 (BSS) sieve and added to step 2.

4. The blend is mixed again for 10 minutes in double cone blender.

5. Magnesium stearate is passed through 44 (BSS) sieve and the final blending was done for 5 minutes.

6. Lubricated blend of step 5 is compressed on 11 mm flat round punch, on 16-station rotary compression machine.

7. The tablets of step 5 are subjected to relative humidity.

8. The tablets of step 7 are vacuum dried.

These tablets had mouth-dissolving time of less than 20 seconds.

### EXAMPLE 2

Simvastatin Mouth Soluble Tablets (5 mg Strength)

| Ingredients | Mg/Unit |
|---|---|
| Simvastatin | 5.0 |
| Butylhydroxyanisole | 0.25 |
| Mannitol | 29.75 |
| Directly compressible lactose | 40.0 |
| L-hydroxypropyl cellulose | 6.0 |
| Sodium bicarbonate | 15.0 |
| Citric acid (anhydrous) | 15.0 |
| Aspartame | 5.0 |
| Pineapple Flavour | 2.0 |
| Magnesium stearate | 2.0 |
| Total | 120.00 |

Process:

1. Simvastatin (BHA-treated), directly compressible lactose, L-hydroxypropyl cellulose, mannitol, pineapple flavour, aspartame, sodium bicarbonate (preheated at 80° C. for 1 hour), are sifted through 44 BSS sieve.

2. The blend of step 1 is mixed for 10 minutes in double cone blender.

3. Citric acid (anhydrous) is sifted through 100 BSS sieve (preheated at 80° C. for 1 hour) and mixed with the blend of step 2; the blend is then mixed for 10 minutes in a double cone blender.

4. The blend of step 3 is lubricated with magnesium stearate (sifted through sieve 44 BSS) by mixing for five minutes in a double cone blender.

5. The blend of step 4 is compressed using 7 mm standard concave punch.

6. The tablets of step 5 are subjected to relative humidity.

7. These tablets are then vacuum dried.

These tablets had a mouth dissolving time of less than 20 seconds.

### EXAMPLE 3

Olanzapine Mouth Soluble Tablets (5 mg Strength)

| Ingredients | Mg/Unit |
|---|---|
| Olanzapine USP | 5.0 |
| Mannitol | 30 |
| Directly compressible Lactose | 35 |
| Croscarmellose sodium | 4 |
| Sodium bicarbonate | 8 |
| Citric acid (anhydrous) | 12 |
| Aspartame | 3 |
| Orange Flavour | 2 |
| Magnesium stearate | 1 |
| Total | 100.00 |

Process:

1. Olanzapine, directly compressible lactose, croscarmellose sodium, mannitol, orange flavour, aspartame, sodium bicarbonate (preheated at 80° C. for 1 hour), are sifted through 44 BSS sieve.

2. The blend of step 1 is mixed for 10 minutes in double cone blender.

3. Citric acid anhydrous (preheated at 80° C. for 1 hour) is sifted through 100 BSS sieve and mixed with the blend of step 2; the blend is then mixed for 10 minutes in a double cone blender.

4. The blend of step 3 is lubricated with magnesium stearate (sifted through sieve 44 BSS) by mixing for five minutes in a double cone blender.

5. The blend of step 4 is compressed using 6.4 mm flat round punch.

6. The tablets of step 5 are subjected to relative humidity.

7. These tablets are then vacuum dried.

These tablets had a mouth dissolving time of less than 20 seconds.

### EXAMPLE 4

Rofecoxib Mouth Soluble Tablets (50 mg Strength)

| Ingredients | Mg/Unit |
|---|---|
| Rofecoxib | 50 |
| Polyvinylpyrrolidone | 0.375 |
| Water | qs |
| Mannitol | 168.625 |
| Microcrystalline cellulose | 50 |
| L-hydroxypropyl cellulose | 20 |
| Sodium bicarbonate | 48 |
| Citric acid (anhydrous) | 40 |
| Aspartame | 12.0 |
| Colloidal Silicon dioxide | 2.0 |
| Mango Flavour | 4.2 |
| Banana Flavour | 0.8 |
| Magnesium stearate | 4.0 |
| Total | 400.00 |

Method

1. Rofecoxib (granulated), mannitol, sodium bicarbonate (preheated at 80° C. for 1 hour), L-hydroxypropyl cellulose, microcrystalline cellulose, Aspartame, colloidal silicon dioxide, Mango flavour, Banana flavour are sifted through 44 BSS sieve.

2. The blend is mixed for 10 minutes in a double cone blender.

3. Citric acid bicarbonate (preheated at 80° C. for 1 hour) is sifted through 100 (BSS) sieve and added to step 2.

4. The blend is mixed again for 10 minutes in double cone blender.

5. Magnesium stearate is passed through 44 (BSS) sieve and the final blending was done for 5 minutes.

6. Lubricated blend of step 5 is compressed on 11 mm flat round punch, on 16-station rotary compression machine.

7. The tablets of step 6 are subjected to a temperature of 80° C. for 30 minutes and the kept at ambient temperature for 8 hours.

8. The tablets of step 7 are vacuum dried.

These tablets had mouth-dissolving time of less than 20 seconds.

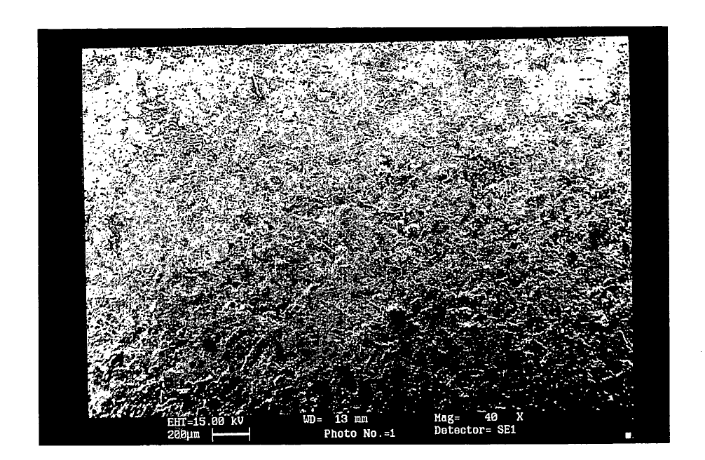

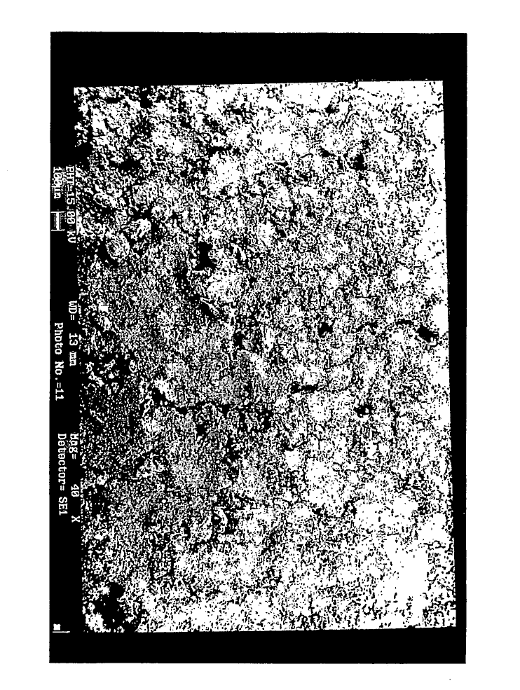

Scanning Electron micrographs (FIGS. 1 & 2) of the rofecoxib tablets prepared using composition of Example 1 clearly show the pore formation in the tablets after the moisture activation.

While the present invention has been described in terms of its specific embodiments, certain modifications and equivalents will be apparent to those skilled in the art and are intended to be included within the scope of the present invention.

1. A process for preparing fast dissolving dosage form, for oral administration, comprising the steps of
a) compressing a blend comprising a pharmaceutical active ingredient and effervescent mixture comprising an acid source and a base to produce a tablet, and
b) subjecting said tablet to moisture activation.

2. The process according to claim 1 wherein the dosage form is a tablet.

3. The process according to claim 2 wherein the tablet dissolves in the mouth.

4. The process according to claim 3 wherein the tablet dissolves in the mouth in less than 20 seconds.

5. The process according to claim 1 wherein one or more pharmaceutical active ingredients is selected from the group consisting of antacids, non-steroidal anti-inflammatory drugs, steroidal anti-inflammatory drugs, anti-psychotic drugs, hypnotic drugs, antiepileptic drugs, antiparkinsonism drugs, hormone drugs, analgesic drugs, serotonin 5HT receptor antagonists, diuretic drugs, coronary vasdilators, H2 receptor antagonists, antiarrthythmic drugs, cardiotonic drugs, calcium antagonists, antihistaminic drugs, antibiotics, antitumor drugs, antidiabetic drugs, central nervous system acting drugs, antispasmodic drugs, antihyperlipidemic drugs, bronchodilators, α-adrenergic receptor blockers, osteoporosis treating drugs, antifungal drugs, antiviral drugs, drugs for erectile dysfunction and antidepressant.

6. The process according to claim 5 wherein the pharmaceutical active ingredient is selected from the group consisting of omeprazole, rofecoxib, nimesulide, betamethasone, olanzapine, alprazolam, sodium valproate, levodopa, progestin, aspirin, ondansetron, sulphamethoxazole, nitroglycerin, ranitidine hydrochloride, pindolol, digitoxin, diltiazem hydrochloride, fexofenadine hydrochloride, doxycycline, actinomycin, metformin, allopurinol, loratadine, quinapril, indeloxazine hydrochloride butyscopolamine, simvastatin, salbutamol, tamsulosin hydrochloride, sodium alderonate, fluconazole, lamivudine, sildenafil and sertraline.

7. The process according to claim 1 wherein the acid source is an acid, anhydride or an acid salt.

8. The process according to claim 7 wherein the acid is selected from the group consisting of citric, tartaric, malic, fumaric, adipic, succinic and alginic acids.

9. The process according to claim 8 wherein the acid is citric acid.

10. The process according to claim 7 wherein the acid salt is selected from the group consisting of dihydrogen phosphate, disodium dihydrogen phosphate and citric acid salts.

11. The process according to claim 1 wherein the base is a solid carbonate.

12. The process according to claim 11 wherein the solid carbonate is selected from the group consisting of sodium carbonate, sodium bicarbonate, potassium carbonate, potassium bicarbonate, magnesium carbonate, sodium glycine carbonate, L-Lysine carbonate, arginine carbonate and amorphous calcium carbonate.

13. The process according to claim 12 wherein the solid carbonate is sodium bicarbonate.

14. The process according to claim 1 wherein the amount of effervescent mixture is from 1 to 35% by weight of the total composition.

15. The process according to claim 14 wherein the amount of effervescent mixture is 15-20% by weight of the total composition.

16. The process according to claim 1 wherein the moisture activation is done by exposing the tablets to controlled humidity or controlled heating.

17. The process according to claim 16 wherein the moisture activation is done by exposing the tablets to controlled humidity.

18. The process according to claim 16 wherein the moisture activation is done by exposing the tablets to controlled heating.

19. The process according to claim 17 wherein the tablets are exposed to controlled humidity by keeping the tablets in relative humidity chamber.

20. The process according to claim 19 wherein the relative humidity chamber has a relative humidity of 20 to 100% at a temperature of about 25° to 90° C.

21. The process according to claim 20 wherein the relative humidity is 50 to 75% at a temperature of about 30° to 50° C.

22. The process according to claim 21 wherein the relative humidity is 75% at a temperature of about 40° C.

23. The process according to claim 17 wherein the tablets are exposed to controlled humidity for up to 2 weeks.

24. The process according to claim 17 wherein the tablets are exposed to controlled humidity for up to 24 hours.

25. The process according to claim 18 wherein the tablets are exposed to controlled heating under vacuum.

26. The process according to claim 1 further comprising the step of removing the moisture by subjecting the tablets to vacuum.
